(19) United States
(12) Patent Application Publication
(10) Pub. No.: US 2018/0052964 A1
(43) Pub. Date: Feb. 22, 2018

(54) Digital Health Ecosystem

(71) Applicant: Resolve Digital Health Inc., Concord (CA)

(72) Inventor: Rob ADELSON, Vaughan (CA)

(73) Assignee: Resolve Digital Health Inc., Concord (CA)

(21) Appl. No.: 15/671,824

(22) Filed: Aug. 8, 2017

Related U.S. Application Data
(60) Provisional application No. 62/375,615, filed on Aug. 16, 2016.

(51) Int. Cl. G06F 19/00; A61M 15/00; A61M 11/00

(52) U.S. Cl. CPC G06F 19/3406; G06F 19/3462; G06F 19/3418; G06F 19/322; A61M 15/002; A61M 11/001; A61M 2205/52; A61M 2205/50; A61M 2205/581; A61M 2205/582; A61M 2205/583; A61M 2205/3584; A61M 2205/33

(57) ABSTRACT

An aspect of the present disclosure relates to an integrated digital health system that includes a smart dosage delivery device connected to a smart appliance and/or a cloud/server. The integrated digital health system primarily includes a smart dosage delivery device that can offer dosing and/or patient interactivity and can connect to a smart appliance and/or a cloud/server to transmit information pertaining to dosage and various health related parameters. The smart appliance and/or cloud/server can include a big data application that can offer analytics and visualization tools to gain insights into customer and patients needs and wants.

### TECHNICAL FIELD

The present disclosure generally relates to the field of digital health. In particular, the present disclosure relates to an integrated system that allows a smart dosage delivery device to connect to a big data infrastructure that offers analysis and visualization of the patient related parameters.

### BACKGROUND

Background description includes information that may be useful in understanding the present invention. It is not an admission that any of the information provided herein is prior art or relevant to the presently claimed invention, or that any publication specifically or implicitly referenced is prior art.

A large number of people are using more drugs, especially prescription drugs, over-the-counter medications and herbal or other nutritional supplements, than ever before. This growth in drug intake has been fuelled by many trends: availability of a wider variety of medications, availability of many former prescription medications as over-the-counter medications, growth of direct-to-consumer advertising of prescription medications, and media hype surrounding new medications and herbal supplements. Also, as more sophisticated treatments have been developed for a variety of conditions, patients are often prescribed complex drug regimens involving multiple medications that must be taken at various times and in various combinations throughout the day. Further, the U.S. population is aging, and the elderly often use multiple medications and supplements for a number of problems. People are also taking “lifestyle enhancing” drugs for conditions that they used to “live with”.

The proliferation of medications generally is a contributing factor to the large number of iatrogenic illnesses, i.e., illnesses that are caused by medical care itself, that affect patients every year, including illnesses that lead to hospitalizations. Polypharmacy, the excessive prescription of multiple and interacting drugs, is a serious issue. Medication-related iatrogenic illnesses may be caused by drug overdoses or otherwise incorrect dosages, incorrect medications, harmful drug interactions, missed doses or incorrect timing of doses. These illnesses may result because patients may get confused about their prescriptions, receive incorrectly filled prescriptions, are being forgetful regarding the quantity, type or timing of a dose, or misidentify a particular medication, miss an expiration date, or mix pills in the same container. These problems further delay the delivery of effective treatment for medication-induced illnesses.

The growth of the Internet and the World Wide Web has further empowered patients to seek health care information on their own. It is well-recognized that increasing number of patients are turning to the Internet for health-related information. Many web sites offer general medical information and many others offer full service drug stores. Via the Internet, patients can be active consumers of information rather than passive recipients of information disseminated by more traditional media. With more information, consumers can generally be better patients and better advocates for themselves in the health care marketplace.

In an effort to attain positive health outcomes in a more cost-effective and timely manner, to date, some solutions to help patients manage medications have been developed. Generally, these solutions fall into two categories: (1) Patient Based Products—these products are reminder pill boxes and auto-dispensing mechanisms. They are difficult to program, configured for a very limited number of medications, and too cumbersome to be considered a portable personal device. In particular, they do not easily link into the medical information chain and allow the physicians, pharmacists and/or pharmaceutical companies to interact with the patient, nor do they provide complete medical information. (2) Medical Organization Based Products—these products are database systems run by hospitals, health maintenance organizations (HMO's) or health insurance companies. They are system tied to these large organizations, with significant computer hardware requirements and no portability. Typically, they are accessible only by physician/hospital personnel or pharmacists. In particular, the patient has no ability to control or read information at their discretion. Also, in the pharmaceutical industry, the current monitoring system for drugs includes non-electronic laminates and labels. This method is very time consuming and not useful for the automatic patient compliance and patient data gathering. Currently few electronic indicators are in research level for the tracking and the monitoring of the pharmaceutical product.

In last few years, use of smartphones, tablets and other smart gadgets has become very popular in every age group. People are extensively using health related application on these smart gadgets. It is followed by wearable health sensing device such as glucose sensor, digital blood pressure sensor, walking sensor etc. which gives information about your health using application installed on your smart device. These applications give information about the body physiological conditions like glucose level, calories burnt, blood pressure, correct body posture etc. based on the sensor integrated to the smart device. Digital Health is an evolving industry that allows the collection of data regarding health, medications, lifestyle, wellness and symptoms management and creates a large amount of data that allows customers and patients, as well as the medical community and pharmaceutical companies to gain a better understanding of people's needs and wants, and offer better solutions.

However, in general, these solutions address specific parts of the overall problem and have thus far failed to make a significant impact on the overall healthcare industry. There is therefore an immediate need in the art to develop an integrated system that allows a smart dosage delivery device to connect to a big data infrastructure that offers analysis and visualization tools to gain insights into patients needs and wants.

### OBJECTS OF THE INVENTION

An object of the present disclosure is to provide an integrated digital health system that can be used for multiple health applications or issues.

Another object of the present disclosure is to provide an integrated digital health system that is customizable, which can be altered and/or modified to fit a wide variety of patient requirements.

Another object of the present disclosure is to provide an integrated digital health system that can offer analytical and visualization tools to gain insights into customer and patients' needs and wants.

Another object of the present disclosure is to provide an integrated digital health system that can collect, store and transmit different levels of patient data, such as, pre-dose data, evaluation and correlation with previous health conditions, medications, family history, lifestyle information and the like.

Another object of the present disclosure is to provide an integrated digital health system that can improve patient compliance to treatment by self-participation of patient in management.

Another object of the present disclosure is to provide an integrated digital health system that is user friendly.

Another object of the present disclosure is to provide an integrated digital health system that is economically viable.

Another object of the present disclosure is to provide an integrated digital health system that can collect and disseminate valuable patient information to third parties for further research and development in inhalable medications.

Another object of the present disclosure is to provide an efficient and cost-effective integrated digital health system.

### SUMMARY

The present disclosure generally relates to the field of digital health. In particular, the present disclosure relates to an integrated system that allows a smart dosage delivery device to connect to a big data infrastructure that offers analysis and visualization of the patient related parameters.

An aspect of the present disclosure provides an integrated digital health system including a smart dosage delivery device configured to collect a patient data; and any or a combination of a smart appliance and a server, operatively coupled with said smart dosage delivery device and configured with an integrated big data application, wherein the smart dosage delivery device is further configured to encrypt the patient data and transmit the patient data to said any or a combination of the smart appliance and the server, and wherein said integrated big data application is configured to perform any or a combination of (a) conversion of the encrypted patient data into a readable form and (b) analysis of the patient data.

In an embodiment, the smart dosage delivery device is selected from a group including a Metered Dose Inhaler (MDI), a spray, and an oral dosage delivery device. In an embodiment, the smart dosage delivery device is configured with one or a plurality of sensors and a processor operatively coupled therewith to collect and/or monitor said patient data. In an embodiment, the smart dosage delivery device is further configured with any or a combination of a gyroscope, an accelerometer and a geographic tracking device. In an embodiment, the patient data comprises any or a combination of (a) patient demographics; (b) patient medication data comprising any or a combination of patient medication schedule, total number of doses in a container coupled with the smart dosage delivery device, number of doses dispensed by the smart dosage delivery device, time of dispensation of dose and number of doses remaining in the container; (c) location of the smart dosage delivery device; and (d) data pertaining to alleviation or aggravation of patient symptoms after administration of the dose. In an embodiment, the smart dosage delivery device is further configured with a medication reminding means selected from any or a combination of an audio indicator, a visual indicator, an audio-visual indicator and a haptic feedback provider.

In an embodiment, the smart dosage delivery device is operatively coupled to said any or a combination of the smart appliance and the server via any or a combination of Wi-Fi, Bluetooth and GSM. In an embodiment, the smart appliance comprises any or a combination of a personal computer, a PDA, a tablet, an ipad, a mobile phone and a smartphone. In an embodiment, integrated digital health system further comprises a repository, operatively coupled to said server and configured to store the patient data. In an embodiment, the smart dosage delivery device is configured to associate the patient demographics with any or a combination of the patient medication data, the location of the smart dosage delivery device and the data pertaining to alleviation or aggravation of patient symptoms after administration of the dose. In an embodiment, the smart dosage delivery device is Metered Dose Inhaler (MDI). In an embodiment, the any or a combination of the smart appliance and the server is further configured to calculate a medication dose based on the patient data. In an embodiment, the calculated medication dose information is transmitted to the smart dosage delivery device and wherein, the smart dosage delivery device is configured to administer a medicine to the patient based on the calculated medication dose.

### BRIEF DESCRIPTION OF THE DRAWINGS

FIG. 1 illustrates an exemplary schematic of an integrated digital health system in accordance with embodiments of the present disclosure.

FIG. 2 illustrates an exemplary flow diagram showing the steps executed in the integrated digital health system in accordance with an embodiment of the present disclosure.

FIG. 3 illustrates an exemplary schematic of the integrated digital health system incorporating/integrated with adaptive dosing mechanism in accordance with embodiments of the present disclosure.

FIG. 4 illustrates an exemplary smart appliance or remote server or any other computing device in which or with which embodiments of the present invention may be utilized in accordance with embodiments of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Various terms as used herein are shown below. To the extent a term used in a claim is not defined below, it should be given the broadest definition persons in the pertinent art have given that term as reflected in printed publications and issued patents at the time of filing.

The present disclosure generally relates to the field of digital health. In particular, the present disclosure pertains to an integrated digital health system that allows a smart dosage delivery device to connect to a big data infrastructure that offers analysis and visualization of the patient related parameters.

An aspect of the present disclosure provides an integrated digital health system including a smart dosage delivery device configured to collect a patient data; and any or a combination of a smart appliance and a server, operatively coupled with said smart dosage delivery device and configured with an integrated big data application, wherein the smart dosage delivery device is further configured to encrypt the patient data and transmit the patient data to said any or a combination of the smart appliance and the server, and wherein said integrated big data application is configured to perform any or a combination of (a) conversion of the encrypted patient data into a readable form and (b) analysis of the patient data.

In an embodiment, the smart dosage delivery device is selected from a group comprising a Metered Dose Inhaler (MDI), a spray, and an oral dosage delivery device. In an embodiment, the smart dosage delivery device is Metered Dose Inhaler (MDI).

In an embodiment, the smart dosage delivery device is configured with one or a plurality of sensors and a processor operatively coupled therewith to collect and/or monitor said patient data.

In an embodiment, the smart dosage delivery device is further configured with any or a combination of a gyroscope, an accelerometer and a geographic tracking device to obtain location data pertaining to the smart delivery device and associate the accelerometer data and/or gyroscope data with other patient data (viz. patient demographics, patient medication data and data pertaining to alleviation or aggravation of patient symptoms after administration of the dose). In an embodiment, the smart dosage delivery device is configured to connect with the external device(s), including PDA and the likes operatively coupled to geographic information system (GIS), to obtain geographical location information pertaining to the patient (alternatively and synonymously referred to as “the user” throughout the present disclosure). In an embodiment, the smart dosage delivery device is configured to associate the geographical location information of the user, obtained from the external device(s), with the patient/user data.

In an embodiment, the patient data includes any or a combination of (a) patient demographics; (b) patient medication data comprising any or a combination of patient medication schedule, total number of doses in a container coupled with the smart dosage delivery device, number of doses dispensed by the smart dosage delivery device, time of dispensation of dose and number of doses remaining in the container; (c) location of the smart dosage delivery device; and (d) data pertaining to alleviation or aggravation of patient symptoms after administration of the dose, but not limited thereto.

In an embodiment, the smart dosage delivery device is further configured with a medication reminding means selected from any or a combination of an audio indicator, a visual indicator, an audio-visual indicator and a haptic feedback provider.

In an embodiment, the smart dosage delivery device is operatively coupled to said any or a combination of the smart appliance and the server via any or a combination of Wi-Fi, Bluetooth and GSM.

In an embodiment, the smart appliance includes any or a combination of a personal computer, a PDA, a tablet, an ipad, a mobile phone and a smartphone, but not limited thereto.

In an embodiment, integrated digital health system further incudes a repository, operatively coupled to said server and configured to store the patient data.

In an embodiment, the smart dosage delivery device is configured to associate the patient demographics with any or a combination of the patient medication data, the location of the smart dosage delivery device and the data pertaining to alleviation or aggravation of patient symptoms after administration of the dose.

In a preferred embodiment, data collected by the smart dosage delivery device, the patient data, is transmitted or transferred to a remote database or server/cloud. The server/remote database may or may not be located centrally relative to the smart dosage delivery device. A “remote database” may refer to one or more congruent and/or distributed databases, such as, for example, also including one or more sets of congruently inter-related databases. According to one or more preferred embodiments of the present disclosure, the database(s) are provided remotely of the smart dosage delivery device.

Preferably, this remote database/server constantly receives collected patient data from one or more smart dosage delivery devices and stores the collected patient data in the database. In an embodiment, confidentiality of patient data transmitted and received between smart dosage delivery device and remote database can be maintained via means of encryption and decryption. The cryptographic application may be a public key cryptographic system or symmetric key (private key) system, or a combination of the two or any other suitable cryptographic system, as known to a person skilled in the art. In an embodiment, the smart dosage delivery device performs encryption of the patient data before transmission thereof to the server/remote database.

The collected patient data is preferably subject to analysis which involves the application of different algorithms to detect trends and/or patterns within the collected data. Analysis of the collected data may be performed by the big data application configured onto the server/cloud. In an alternative embodiment, the analysis of the collected data is performed by an external device and/or an analysis unit operatively coupled with the server. Persons skilled in the art, however, will appreciate that the analysis of patient data may also be performed local to the smart dosage delivery device. The collected data is preferably analyzed for additional information associated with the patient.

A person skilled in the relevant art would generally understand that a reference to “Internet of Things (IoT)” refers to networked or interconnected objects, typically, but not limited to, everyday objects, more technically purposed objects (i.e. medical devices) and devices. It is described as a self-configuring wireless network of sensors whose purpose would be to interconnect all such connected devices. The concept is attributed to the former Auto-ID Center, founded in 1999, based at the time at the Massachusetts Institute of Technology (“MIT”).

Patient data collected by the smart dosage delivery device may be used for various applications, including but not limited to allowing healthcare providers to enhance user treatment by analyzing the user data and implementing appropriate strategies to enhance the efficacy of the substance or overall treatment. Thus, the smart dosage delivery device allows a healthcare provider to enhance patient outcomes. Patient data may be used in a clinical setting to determine efficacy and other treatment variables on a more generalized basis (e.g. as anonymous population data). Patient data can also be used to enhance individual user outcomes. For example, physicians can provide patients with a sample of an herbal and/or therapeutic substance and obtain feedback (i.e. patient data) from the smart dosage delivery device to allow the physician to evaluate the effectiveness of the substance. Additional exemplary applications of the method and smart dosage delivery device of the present disclosure may include: (a) pain relief and other symptom relief for medical cannabis patients; (b) naturopath and other alternative health care practitioner prescribed herbal remedies for symptom relief; (c) sleep support, anti-emetic, mood control by vaporizing herbs and related essential oils; and/or (d) a research tool for major universities and pharmaceutical companies to be used for testing alternative delivery mechanisms for medicine. In an embodiment, the smart dosage delivery device can be used to inhale substances derived from a cannabis, or marijuana, plant.

In an embodiment, the smart dosage delivery device can be configured with a GUI, a gyroscope/accelerometer, a vibrational motor or other haptic feedback providing means, one or more LEDs, a speaker/reader and a microphone for collecting and/or presenting patient data to the user. In an embodiment, the smart dosage delivery device is configured to provide medication reminder to the user by providing signal/alert in any or a combination of forms of lighting the one or a plurality of LEDs, vibrating the smart dosage delivery device or generating a sound. In an alternative embodiment, the GUI, vibrational motor, LED and speaker can be configured with the smart appliance to generate alerts for the user and/or caretakers when the medication is not taken appropriately. In an embodiment, the alerts issued to the user or caretakers of the patient can be through any communication channel such as a phone call, IVR, SMS, MMS, or any other messaging system. The alerts can also be issued to the user or caretakers of the patient as an alarm or a screen pop on his/her smart appliance. However, it is to be appreciated that any other communication channel, as known to a person skilled in the art, can be utilized for the aforementioned purpose without departing from the scope and spirit of the present disclosure.

In an embodiment, the smart dosage delivery device can be configured with a means to input the feedback of the patient relating to the dosage administration or efficacy of the medication administered. In an embodiment, the user can use gestures (e.g. direction of smart dosage delivery device to indicate his/her feeling or status) in order to provide feedback to the smart dosage delivery device (e.g. patient data).

In an embodiment, the integrated digital health system of the present disclosure is configured for use with a communication network. The communication network may include satellite networks (e.g. GPS), terrestrial wireless networks, and the Internet. The communication of data between a device subsystem, an external device subsystem and/or an accessory device subsystem may also be achieved via one or more wired means of transmission (e.g. docking the smart docking device in a base station of the external device subsystem), or other physical means (e.g. a Universal Serial Bus cable and/or flash drive) of transmission. Persons having ordinary skill in the art will appreciate that the system includes hardware and software.

In a preferred embodiment, the smart dosage delivery device and/or smart appliance may comprise a hardware and/or software application that allows for the receipt or transmission of data that has the capability to use the 802.11 protocol, Bluetooth communication and/or another linkage. For example, cellular communication and/or the communication network may be used. Additional hardware and/or software applications may: (i) be enacted upon associating the device with a user; (ii) connect wirelessly to one or more processor(s) of an external device (e.g. via Bluetooth, Wi-Fi and/or another linkage) for the exchange of data; and/or (iii) store data (e.g. patient data) in one or more databases for subsequent transmission and/or analysis.

In an embodiment, the system is configured to collect patient data including, among other information, patient demographics, time, date and number of doses administered and the likes. By collecting patient data, the smart appliance and/or cloud based application enables analysis and control of therapy by authorized parties (e.g. healthcare professionals, law enforcement, governments, etc.). In one embodiment, the smart dosage delivery device receives pods containing a pre-packaged dose of the medical substance, and the smart dosage delivery device is able to collect patient data and interface with external devices and/or remote databases. In an embodiment, the integrated digital health system is advantageous for patients, healthcare professionals, regulators, and/or administrators.

In an embodiment, the server is configured with an integrated big data application. The server can receive the patient data directly from the smart dosage delivery device and/or from the smart appliance. In an alternative embodiment, the integrated big data application can be configured on the smart appliance and/or the cloud based application. The integrated big data application can be configured to receive, store, and/or analyze patient data, including symptoms, pain relief, appetite, physical energy, exercise, diet, and use of (including therapeutic and adverse effects of) other medications. The patient data is securely encrypted with controlled access.

In some embodiments, the integrated big data application provides healthcare professionals with a comprehensive report generated based on the patient data collected by the smart dosage delivery device and/or data from external devices (including third party monitoring devices such as smart blood pressure machine, glucose monitoring, etc.). Preferably, the integrated big data application is compatible with various operating systems including, but not limited to iOS, Android, Windows, and Internet web browsers (e.g. Internet Explorer, Firefox, etc.).

In an embodiment, the integrated big data application is capable of conducting a variety of analyses (and creates a number of reports) based on the patient data and/or data from external devices including, but not limited to: Dosing—measures and/or monitors dosing for users; Dose effective tracking—collects user feedback after each dose; Symptom tracking—monitors all patient symptoms; Medication tracking—monitors all medications that patients are using or have used; Reporting—creates graphs and trend chart(s) and easy to comprehend infographics on patient(s) or users and may be compared with patient(s) or users with similar health issues; Lifestyle program—provides a unique lifestyle program and measures patient involvement and results; Retail—linked into the retail environment to ensure that patients can acquire refills and new products; Supply chain management—links into cultivation network to ensure that growers and distributors have the information they need to supply patients with the best products; and/or Support—allows patients or users to connect to the people most important to them and encourages interaction and support. In preferred embodiment, the integrated big data application collects data, including patient data, on: all of the medications taken, the effectiveness of each dose, how a patient or user feels, exercise, diet, mindfulness, support, and information from other, or external, devices (e.g. weight, blood pressure, blood sugar, cholesterol, etc.). In an embodiment, the integrated big data application allows guiding the users on how to use the smart dosage delivery device, coaches about one's condition and aids in following a prescribed regimen by learning from the usage patterns recorded by the sensor.

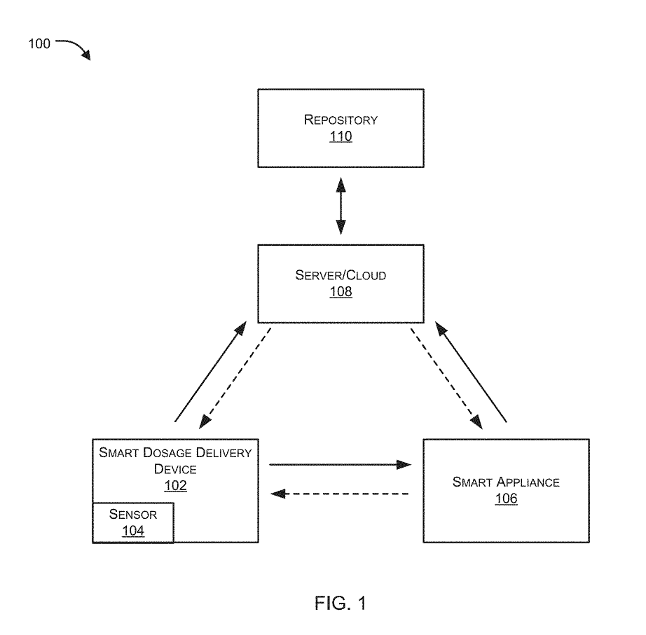

FIG. 1 illustrates an exemplary schematic of an integrated digital health system in accordance with embodiments of the present disclosure. In an embodiment, the smart dosage delivery device, as shown as 102, can be any dosing device, as known to a person skilled in the art. In an exemplary embodiment, the smart dosage delivery device 102 is selected from a Metered Dose Inhaler (MDI), spray, oral dosage delivery system and the like configured with integrated sensor(s), as shown as 104, to measure the dosage, and a transmission mechanism to transmit said information. In an embodiment, the smart dosage delivery device is MDI. In an embodiment, the smart dosage delivery device 102 can have custom built circuit boards. In another embodiment, smart dosage delivery device with custom built circuit boards can have arrangement for dose measurement. In still another embodiment, it can also have a means of connectivity including, but not limited to Wi-Fi, Bluetooth, GSM and the like. In an embodiment, the smart device 102 can have a sensor 104 that can wirelessly transmit the data to a smart appliance 106. The sensor can use any suitable transmission protocol, such as Wi-Fi, bluetooth, GSM or the like, to transmit the dosage data.

In an embodiment, smart dosage delivery device 102 can establish connection with a smart appliance 106 by any means of connectivity including, but not limited to Wi-Fi, Bluetooth, GSM. In another embodiment, smart dosage delivery device 102 can be physically coupled to smart appliance 106 by any wired connection. In an embodiment, smart appliance 106 can be a personal computer or a tablet or an ipad or a mobile phone or a smartphone, such as an iPhone, android phone, windows phone, or a blackberry phone, which can further establish a connection with a server and/or cloud based application, as shown as 108, via any means of interconnection including but not limited to NFC, RFID, sensor, wifi and the like. In an exemplary embodiment, server and/or cloud based application 108 can include one or more databases (repository), as shown as 110, which allows for storage of medical information. The server may include an HTTP/HTTPS server sending and receiving HTTP/HTTPS messages in order to provide web browsing user interfaces to client web browsers. The server may be implemented in one or more actual servers as known in the art, and may send and receive medial information, user supplied information, or configuration data, among other data, that may be transferred to, read from, or stored in the repository. The repository may include a relational database such as an SQL database, or fixed content storage system, used to store medical information or any other configuration or administration information required to implement the services. The repository may include one or more physical servers, databases, or storage devices that are necessary to implement the service's storage requirements.

In an embodiment, the smart appliance 106 can then send the dosage data to a server/cloud. Certain embodiments provide Web portal applications for data presentation to patients. A Web-based portal can provide an adaptive and proactive experience for users including matching technology/tools, education/information, and guided feedback based upon a patient's specific personality and lifestyle assessment, for example.

In an embodiment, the smart appliance 106 and/or the cloud/server 108 can include an integrated big data application that can store the data safely and securely and in accordance with local data privacy regulations. In an embodiment, the integrated big data application can then analyze and interpret the patient data. In another embodiment, the analyzing and interpretation tools can convert the encrypted data from the patient into a readable form.

In an embodiment, standards-based components are utilized for information exchange. These components can conform to the latest healthcare industry technical standards. In situations where clinical systems, which will be sending patient information to participating registries and repositories, cannot communicate according to one or more industry standards, messages can be transformed into messages complying with one or more applicable standards prior to being added to the registries or repositories. In another embodiment, the integrated big data application can offer analytic and visualization tools to gain insights into patients' needs and wants.

In another embodiment, the smart appliance 106 and/or server/cloud 108 can then selectively disseminate the data to multiple data sources, including but not limited to, payers, financial institutions, EMR systems, practice management systems, claims/prescription databases, pharmaceutical companies, physician/hospital portals, pharmacy benefit management (PBM) and the like, on request. In another embodiment, the smart appliance 106 and/or cloud 108 can then selectively disseminate the physiological data to multiple data sources even without request. In another embodiment, the information can be shared using any suitable wireless transmission protocol, such as Wi-Fi, bluetooth, GSM and the like.

Embodiments of the present invention may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

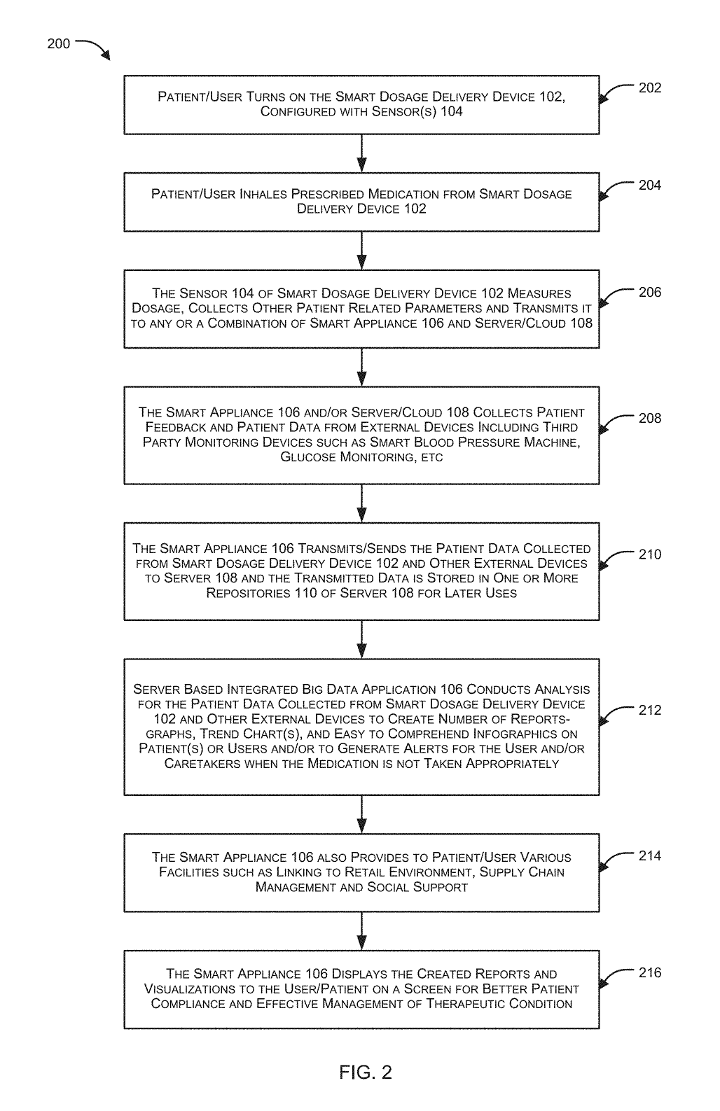

FIG. 2 illustrates an exemplary flow diagram showing steps executed in the integrated digital health system in accordance with an embodiment of the present disclosure. As illustrated, at step 202, a patient or user can turn on the smart dosage delivery device 102, configured with sensor(s) 104, by pressing the power button. At step 204, the patient or user can administer the prescribed medication dispensed by the smart dosage delivery device 102. At step 206, the sensor 104 of the smart dosage delivery device 102 can measure dosage, collect other patient related parameters and transmit them to any or a combination of the smart appliance 106 and the server/cloud 108. At step 208, the smart appliance 106 and/or the server/cloud 108 collect patient feedback and patient data from external devices, which include but not limit to, third party monitoring devices, such as, smart blood pressure machine, glucose monitoring machine, and the like. At step 210, the smart appliance 106 can transmit/send the patient data, collected from both smart dosage delivery device 102 and from external devices, to server 108 and the transmitted data is stored securely in one or more repository/database 110 of the server 108, for later uses. In an embodiment, patient data collected by the smart dosage delivery device 102 may be used for various applications, including but not limited to allowing healthcare providers to enhance user treatment by analysing the user data and implementing appropriate strategies to enhance the efficacy of the substance or overall treatment. Thus, the smart dosage delivery device allows a healthcare provider to enhance patient outcomes. Patient data may be used in a clinical setting to determine efficacy and other treatment variables on a more generalized basis (e.g. as anonymous population data). Patient data can also be used to enhance individual user outcomes. For example, physicians can provide patients with a sample of an herbal and/or therapeutic substance and obtain feedback (i.e. patient data) from the smart dosage delivery device to allow the physician to evaluate the effectiveness of the substance. Additional exemplary applications of the method and smart dosage delivery device 102 of the present disclosure may include: (a) pain relief and other symptom relief for medical cannabis patients; (b) naturopath and other alternative health care practitioner prescribed herbal remedies for symptom relief; (c) sleep support, anti-emetic, mood control by vaporizing herbs and related essential oils; and/or (d) a research tool for major universities and pharmaceutical companies to be used for testing alternative delivery mechanisms for medicine. At step 212, the server based integrated big data application 106 can conduct analysis of the patient data, collected from both smart dosage delivery device 102 and from external devices, to create a number of reports—graphs, trend charts and easy-to-comprehend infographics on patient/user. In an embodiment, the smart dosage delivery device generates alerts for the user and/or caretaker(s), especially when the medication is not taken appropriately. In an embodiment, the alerts issued to the user or caretakers of the patient can be through any communication channel such as a phone call, IVR, SMS, MMS, or any other messaging system. The alerts can also be issued to the user or caretakers of the patient as an alarm or a screen pop on his/her smart appliance. At step 214, the smart appliance 106 can also provide to the user/patient, various facilities which include but are not limited to, Lifestyle program which provides a unique lifestyle program and measures patient involvement and results; linkage to retail environment to ensure that patients can acquire refills and new products; Supply chain management—links into cultivation network to ensure that growers and distributors have the information they need to supply patients with the best products; and/or Support—allows patients or users to connect to the people most important to them and encourages interaction and support. In an embodiment, the patient or the user can choose what kind of and amount of information he/she wants to share with the society. In another embodiment, the patient or customer allows only a specific amount of data to be shared with others. In an embodiment, the information can be shared with the multiple parties. In another embodiment, the multiple parties can include but not limited to any organisation, medical college, health industry etc. In still another embodiment, the third party can utilize this information for research, business, development or production of health goods. At step 216, the smart appliance 106 can display the created reports and visualizations to the user/patient on a screen for better patient compliance and effective management of therapeutic condition(s).

Another aspect of the present disclosure provides to the integrated digital health system that allows the user to control the amount/extent of information (patient data) shared with other users. In an embodiment, the patient or the customer can choose what kind of and amount of information he/she wants to share with the society. In another embodiment, the patient or customer allows only a specific amount of data to be shared with others. In an embodiment, the information can be shared with the multiple parties. In another embodiment, the multiple parties can include but not limited to any organisation, medical college, health industry etc. In still another embodiment, the third party can utilize this information for research, business, development or production of health goods. In an embodiment, sharing of patient or customer information can be governed by laws including privacy laws. In another embodiment, the integrated system can share information under security and privacy policies. In another embodiment, the integrated system does not share any personal information of the patient or customer. In still another embodiment, the personal information can include but not limited to name, location, number etc. In an embodiment, the integrated digital health system can include an administration unit. In another embodiment, the administrator unit can manage or co-ordinate between the administrators, managers, patient or customer, medical practitioners, third parties etc.

Another aspect of the present disclosure provides an integrated digital health system incorporated/integrated with adaptive dosing mechanism(s), wherein any or a combination of the server/cloud and the smart appliance is configured to calculate the dosage (medication dose required to be administered to the patient) based on the patient data. In an embodiment, any or a combination of the server/cloud and the smart appliance is configured to transmit the calculated dosage to the smart dosage delivery device. In an embodiment, the smart dosage delivery device is configured to administer the medication to the patient based on the calculated medication dose.

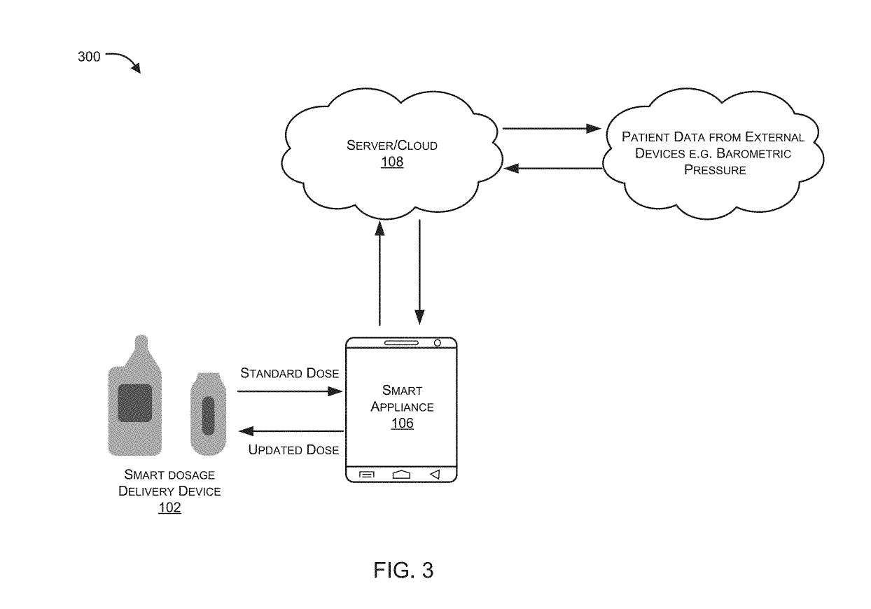

FIG. 3 illustrates an exemplary schematic of the integrated digital health system 300 incorporating and/or integrated with an adaptive dosing mechanism in accordance with an embodiment of the present disclosure. In an embodiment, the integrated digital health system includes a smart dosage delivery device 102, and any or a combination of a smart appliance 106 and a server/cloud 108, operatively coupled with said smart dosage delivery device 102 and configured to collect the patient data (e.g. patient feedback, data pertaining to alleviation or aggravation of patient symptoms, patient data collected from other smart delivery devices). Accordingly, the integrated digital health system incorporated/integrated with adaptive dosing mechanism(s) can utilize the patient data to optimize a patient's medication dose for maximum efficacy.

In an exemplary embodiment, the patient feedback received is that the patient has lower levels of pain associated with his/her therapeutic condition on days when he/she exercises in morning. Accordingly, on a subsequent day when the patient has exercised in the morning, the integrated digital health system can adjust his/her medication dose downwards, wherein any or a combination of the server/cloud and the smart appliance calculates the medication dose and transmit the calculated dose to the smart dosage delivery device, which administers the medicine to the patient/user based on the calculated medication dose. In another exemplary embodiment, the patient data collected from external device(s) includes low barometric pressure of the area where the patient is located, which can cause higher levels of sensation of pain to the patient. Accordingly, on a subsequent day when the barometric pressure of the area where the patient is located is low, the integrated digital health system can adjust his/her medication dose upwards. One would appreciate that the integrated digital health system incorporated/integrated with adaptive dosing mechanism(s) can find utility in any other condition, as known to appreciated by a person skilled in the art, without departing from scope and spirit of the present invention.

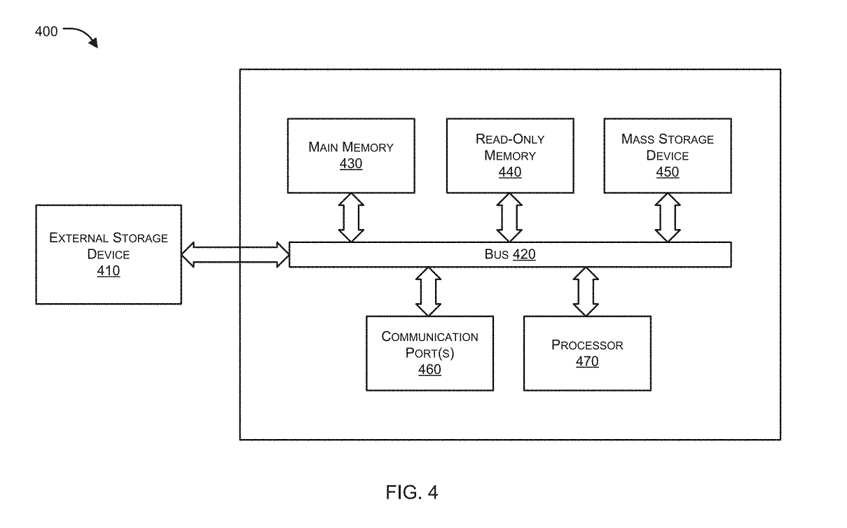

FIG. 4 illustrates an exemplary server or any other computing device in which or with which embodiments of the present invention may be utilized in accordance with embodiments of the present disclosure. In an embodiment, the smart appliance 400 can provide a means through which the patient data enters into the integrated digital health system. A variety of these steps may be performed by hardware components or may be tangibly embodied on a computer-readable storage medium in the form of machine-executable instructions, which may be used to cause a general-purpose or special-purpose processor programmed with instructions to perform these steps. Alternatively, the steps may be performed by a combination of hardware, software, and/or firmware. As shown in the figure, smart appliance 400 includes an external storage device 410, a bus 420, a main memory 430, a read only memory 440, a mass storage device 450, communication port 460, and a processor 470. A person skilled in the art will appreciate that smart appliance 400 may include more than one processor and communication ports. Examples of processor 470 include, but are not limited to, an Intel® Itanium® or Itanium 2 processor(s), or AMD® Opteron® or Athlon MP® processor(s), Motorola® lines of processors, FortiSOC™ system on a chip processors or other future processors. Processor 470 may include various modules associated with embodiments of the present invention. Communication port 460 can be any of an RS-232 port for use with a modem based dialup connection, a 10/100 Ethernet port, a Gigabit or 10 Gigabit port using copper or fiber, a serial port, a parallel port, or other existing or future ports. Communication port 460 may be chosen depending on a network, such a Local Area Network (LAN), Wide Area Network (WAN), or any network to which smart appliance 400 connects. Memory 430 can be Random Access Memory (RAM), or any other dynamic storage device commonly known in the art. Read only memory 440 can be any static storage device(s) e.g., but not limited to, a Programmable Read Only Memory (PROM) chips for storing static information e.g., start-up or BIOS instructions for processor 470. Mass storage 450 may be any current or future mass storage solution, which can be used to store information and/or instructions. Exemplary mass storage solutions include, but are not limited to, Parallel Advanced Technology Attachment (PATA) or Serial Advanced Technology Attachment (SATA) hard disk drives or solid-state drives (internal or external, e.g., having Universal Serial Bus (USB) and/or Firewire interfaces), e.g. those available from Seagate (e.g., the Seagate Barracuda 7200 family) or Hitachi (e.g., the Hitachi Deskstar 7K1000), one or more optical discs, Redundant Array of Independent Disks (RAID) storage, e.g. an array of disks (e.g., SATA arrays), available from various vendors including Dot Hill Systems Corp., LaCie, Nexsan Technologies, Inc. and Enhance Technology, Inc. Bus 420 communicatively couples processor(s) 470 with the other memory, storage and communication blocks. Bus 420 can be, e.g. a Peripheral Component Interconnect (PCI)/PCI Extended (PCI-X) bus, Small Computer System Interface (SCSI), USB or the like, for connecting expansion cards, drives and other subsystems as well as other buses, such a front side bus (FSB), which connects processor 470 to software system. Optionally, operator and administrative interfaces, e.g. a display, keyboard, and a cursor control device, may also be coupled to bus 420 to support direct operator interaction with smart appliance 400. Other operator and administrative interfaces can be provided through network connections connected through communication port 460. External storage device 410 can be any kind of external hard-drives, floppy drives, IOMEGA® Zip Drives, Compact Disc-Read Only Memory (CD-ROM), Compact Disc-Re-Writable (CD-RW), Digital Video Disk-Read Only Memory (DVD-ROM). Components described above are meant only to exemplify various possibilities. In no way should the aforementioned exemplary user computing device or remote server limit the scope of the present disclosure.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### Advantages of the Invention

The present disclosure provides for an integrated digital health system that can be used for multiple health applications or issues.

The present disclosure provides for an integrated digital health system that is customizable, which can be altered and/or modified to fit a wide variety of patient requirements.

The present disclosure provides for an integrated digital health system that can offer analytical and visualization tools to gain insights into customer and patients' needs and wants.

The present disclosure provides for an integrated digital health system that can collect, store and transmit different levels of patient data, such as, pre-dose data, evaluation and correlation with previous health conditions, medications, family history, lifestyle information and the like.

The present disclosure provides for an integrated digital health system that can improve patient compliance to treatment by self-participation of patient in management.

The present disclosure provides for an integrated digital health system that is user-friendly and economically viable.

The present disclosure provides for an integrated digital health system that can collect and disseminate valuable patient information to third parties for further research and development in inhalable medications.

The present disclosure provides for an efficient and cost-effective integrated digital health system.

We claim:

1. An integrated digital health system comprising:
a smart dosage delivery device configured to collect a patient data; and
any or a combination of a smart appliance and a server, operatively coupled with said smart dosage delivery device and configured with an integrated big data application,
wherein the smart dosage delivery device is further configured to encrypt the patient data and transmit the patient data to said any or a combination of the smart appliance and the server, and wherein said integrated big data application is configured to perform any or a combination of (a) conversion of the encrypted patient data into a readable form and (b) analysis of the patient data.

2. The integrated digital health system as claimed in claim 1, wherein said smart dosage delivery device is selected from a group comprising a Metered Dose Inhaler (MDI), a spray, and an oral dosage delivery device.

3. The integrated digital health system as claimed in claim 1, wherein said smart dosage delivery device is configured with one or a plurality of sensors and a processor operatively coupled therewith to collect and/or monitor said patient data.

4. The integrated digital health system as claimed in claim 1, wherein said smart dosage delivery device is further configured with any or a combination of a gyroscope, an accelerometer and a geographic tracking device.

5. The integrated digital health system as claimed in claim 1, wherein said patient data comprises any or a combination of (a) patient demographics; (b) patient medication data comprising any or a combination of patient medication schedule, total number of doses in a container coupled with the smart dosage delivery device, number of doses dispensed by the smart dosage delivery device, time of dispensation of dose and number of doses remaining in the container; (c) location of the smart dosage delivery device; and (d) data pertaining to alleviation or aggravation of patient symptoms after administration of the dose.

6. The integrated digital health system as claimed in claim 1, wherein said smart dosage delivery device is further configured with a medication reminding means selected from any or a combination of an audio indicator, a visual indicator, an audio-visual indicator and a haptic feedback provider.

7. The integrated digital health system as claimed in claim 1, wherein said smart dosage delivery device is operatively coupled to said any or a combination of the smart appliance and the server via any or a combination of Wi-Fi, Bluetooth and GSM.

8. The integrated digital health system as claimed in claim 1, wherein said smart appliance comprises any or a combination of a personal computer, a PDA, a tablet, an ipad, a mobile phone and a smartphone.

9. The integrated digital health system as claimed in claim 1, wherein said smart dosage delivery device is configured to associate the patient demographics with any or a combination of the patient medication data, the location of the smart dosage delivery device and the data pertaining to alleviation or aggravation of patient symptoms after administration of the dose.

10. The integrated digital health system as claimed in claim 1, wherein said integrated digital health system further comprises a repository, operatively coupled to said server and configured to store the patient data.

11. The integrated digital health system as claimed in claim 1, wherein said any or a combination of the smart appliance and the server is further configured to calculate a medication dose based on the patient data.

12. The integrated digital health system as claimed in claim 11, wherein the calculated medication dose is transmitted to the smart dosage delivery device and wherein, the smart dosage delivery device is configured to administer a medicine to the patient based on the calculated medication dose.
